(19) United States
(12) Patent Application Publication
(10) Pub. No.: US 2015/0141515 A1
(43) Pub. Date: May 21, 2015

(54) COMPOSITIONS AND METHODS FOR DELIVERY OF NSAID AND ANESTHETIC

(71) Applicant: FIRSTLINE MEDS, INC., WINSTON-SALEM, NC (US)

(72) Inventors: JAY A. STOLTENBERG, KISSIMMEE, FL (US); SHANE HAINES, HOLLYWOOD, FL (US)

(73) Assignee: FIRSTLINE MEDS, INC., WINSTON-SALEM, NC (US)

(21) Appl. No.: 14/548,352

(22) Filed: Nov. 20, 2014

Related U.S. Application Data
(60) Provisional application No. 61/906,516, filed on Nov. 20, 2013.

(51) Int. Cl. A61K 47/32; A61K 47/18; A61K 47/10; A61K 47/14; A61K 31/196; A61K 31/167

(52) U.S. Cl. CPC A61K 47/32; A61K 31/196; A61K 31/167; A61K 47/10; A61K 47/14; A61K 47/18
USPC 514/567; 206/232

(57) ABSTRACT

The invention relates generally to non-steroidal anti-inflammatory drugs (NSAIDs) and anesthetics, and more particularly, to NSAID and anesthetic compositions, methods of preparing/producing the compositions and methods of delivering, distributing and administering the compositions to patients.

### CROSS-REFERENCE TO RELATED APPLICATIONS

This application claims priority under 35 U.S.C. §119(e) from U.S. provisional patent application No. 61/906,516, entitled “Compositions and Methods for Delivery of NSAID and Anesthetic”, filed on Nov. 20, 2013, the content of which is incorporated herein by reference.

### FIELD OF THE INVENTION

The invention relates generally to non-steroidal anti-inflammatory drugs (NSAIDs) and anesthetics, and more particularly, to NSAID- and/or anesthetic-containing compositions, methods of preparing/producing the compositions and, methods of delivering, distributing and administering the compositions to patients.

### BACKGROUND OF THE INVENTION

NSAIDs are known in the art as a class of drugs that provides at least one of the following effects: (i) analgesic, i.e., pain killing, (ii) antipyretic, i.e., fever-reducing, and (iii) anti-inflammatory. NSAIDs are non-narcotic and therefore, can be useful as a non-addictive alternative to narcotics in certain instances. NSAIDs are usually used for the treatment of acute or chronic conditions where pain and inflammation are present. In general, NSAIDs work on a chemical level to block the effects of enzymes, specifically, COX-1 and COX-2 enzymes. These enzymes have a key role in making prostaglandins, which are a family of chemicals that are produced by the cells of the body. Prostaglandins have several important functions, which include promoting inflammation (which can result in pain and fever) that is necessary for healing, supporting the blood clotting function of platelets and protecting the lining of the stomach from the damaging effects of acid. By blocking the COX enzymes, NSAIDs can reduce the production of prostaglandin, resulting in less swelling and pain, and lower fever. NSAIDs typically include a pharmaceutically active component as well as non-active ingredients. For example, in aspirin, the active component is acetylsalicyclic acid and the inactive ingredients include carnauba wax, corn starch and cellulose.

Anesthetics are also known in the art. They are a class of drugs that cause anesthesia, which is the reversible loss of sensation. Anesthetics are differentiated from analgesics, which relieve pain without eliminating sensation. Anesthetics are generally categorized into two classes which include general anesthetics and local anesthetics. General anesthetics cause a reversible loss of consciousness and local anesthetics cause a reversible loss of sensation for a limited region of the body while maintaining consciousness. Combinations of anesthetics can be employed for their synergistic and additive therapeutic effects.

In general, NSAIDs and anesthetics can be administered to a patient using a variety of delivery mechanisms, such as orally, e.g., by tablet, capsule, liquid, powder and the like; topically or transdermally, e.g., by gels, creams, lotions and the like; and intravenously, e.g., by cocktail injections, immunizations, and the like.

The pharmaceutical industry is a trillion dollar market. Many of the well-known NSAIDs, which include aspirin, ibuprofen and naproxen, are produced by large pharmaceutical drug manufacturers, supplied by distributors to retailers, and made available by the retailers to consumers as over-the-counter medications. Other NSAIDs are available by prescription. Many of the prescription NSAIDs are also produced by large pharmaceutical drug manufacturers, supplied by distributors to retail pharmacies, physicians' offices, hospitals, hospitals' pharmacies and the like, and made available to patients as prescribed drugs wherein a prescription from a medical professional is typically required to obtain the drug.

There is a desire in the art to develop pharmaceutical NSAID and anesthetic compositions that are effective to efficiently treat inflammation and relieve pain, as well as, novel mechanisms as options to traditional models for the production/preparation, distribution, delivery and administration of the compositions to patients.

### SUMMARY OF THE INVENTION

In one aspect, the invention provides a pharmaceutical composition for the treatment of inflammation, comprising a pharmaceutically active ingredient selected from the group consisting of diclofenac and lidocaine, solvent selected from the group consisting of water, alcohol and mixtures thereof, diol and methyl salicylate.

The pharmaceutically active ingredient can be present in an amount of about 0.1 to about 20.0 percent by weight, the water can be present in an amount of about 30% to about 60% by weight, the alcohol can be present in an amount of about 10% to about 50% by weight, the propylene glycol can be present in an amount of about 2% to about 40% by weight and the methyl salicylate can be present in the in an amount of about 0.1% to about 5% by weight.

The composition can further comprise one or more of a penetration enhancer, polymer, skin cooling substance and pH modifying agent. In certain embodiments, the polymer is acrylic acid.

The pharmaceutically active ingredient can be in dry form.

The composition can be in a form for topical administration to a patient in a therapeutically effective amount. The composition can be in the form of a gel.

In another aspect, the invention provides a method of providing a pharmaceutical composition for treating a patient for inflammation. The method includes obtaining a pharmaceutically active ingredient selected from the group consisting of non-steroidal anti-inflammatory drugs, anesthetic, and pharmaceutically acceptable salts thereof and forms thereof, and mixtures thereof, obtaining a solvent, supplying by the supplier to a recipient the pharmaceutically active ingredient and the solvent individually, combining by the supplier or the recipient the pharmaceutically active ingredient and the solvent, at least partially dissolving the pharmaceutically active ingredient in the solvent to form the pharmaceutical composition, placing the pharmaceutical composition in a container, affixing a label to the container to identify the name of the recipient, and offering for sell and selling the container with the label to a patient.

The pharmaceutically active ingredient can be selected from the group consisting of diclofenac, lidocaine, pharmaceutically acceptable salts and forms thereof, and mixtures thereof.

The pharmaceutical composition can include one or more ingredients selected from the group consisting of diol, amine, polymer, methyl salicylate and mixtures thereof. The composition can be in the form of a gel.

The combining step can include the use of equipment that is provided by the supplier to the recipient. The equipment can be purchased, rented or leased by the recipient from the supplier.

The recipient can be selected from the group consisting of a physician, physician's office, pharmacist, pharmacy and hospital.

In yet another aspect, the invention includes a kit for providing a pharmaceutical composition for treating a patient for inflammation. The kit includes one or more containers of pharmaceutically active ingredient selected from the group consisting of non-steroidal anti-inflammatory drugs, anesthetic, and pharmaceutically acceptable salts and forms thereof, and mixtures thereof, one or more containers of solvent, instructions for combining the pharmaceutically active ingredient with the solvent to form the pharmaceutical composition, equipment for use in combining the pharmaceutically active ingredient with the solvent, container for receiving the pharmaceutical composition, labels for affixing to the container to identify the name of a supplier or preparer of the pharmaceutical composition.

### BRIEF DESCRIPTION OF THE DRAWINGS

FIG. 1 is a plot showing in vitro diclofenac sodium flux through human epidermis versus time, in accordance with certain embodiments of the invention.

FIG. 2 is a plot showing in vitro cumulative diclofenac sodium delivery versus time, in accordance with certain embodiments of the invention.

### DETAILED DESCRIPTION OF THE INVENTION

The invention relates to compositions including NSAID, anesthetic or combinations thereof, methods of preparing/producing, these compositions, methods of distributing, delivering and administering these compositions to a patient in a therapeutically effective amount, and uses of the compositions and methods for treating inflammation and/or pain.

As used herein, the term “therapeutically effective amount” means an amount or dosage such that, when administered in a physiologically tolerable composition, is sufficient to achieve an effective systemic concentration or local concentration in tissue to elicit a response in the tissue, system or individual to which it was administered. The term “administering” and the like means to administer a compound or composition systemically or locally, as directly into or onto a target tissue, to a patient whereby the compound or composition positively impacts the tissue to which it is targeted. The compound or composition may be administered by injection, topical administration and oral administration or by other methods alone or in combination with other known techniques. Further, as used herein, the term “pharmaceutically acceptable salts and forms thereof” includes salts and other forms of the present NSAID and anesthetic compounds, e.g., diclofenac and lidocaine, which are suitable for use in pharmaceutical applications. One skilled in the art would easily be able to determine whether a salt form or other form of any given compound is suitable for use as a pharmaceutical.

The compositions of the invention include aqueous, alcohol-based compositions which are effective to deliver active pharmaceutical ingredient generally to a patient or, more particularly, to a target site of a patient. The pharmaceutically active ingredient is selected from NSAID, anesthetic, and pharmaceutically acceptable salts and forms thereof, and mixtures thereof. The compositions can be in a wide variety of forms such as, but not limited to tablets, powders, capsules, liquids, gels, creams, lotions and the like. In certain embodiments, the compositions are in gel form for topical, e.g., transdermal, administration to patients, which results in the pharmaceutically active ingredient(s) penetrating the epidermis of the skin of the patients, causing absorption of the pharmaceutically active ingredient(s) into the dermal layer(s) of the skin, and contacting the site or source of the inflammation.

The compositions of the invention include the pharmaceutically active ingredient which is generally effective to treat pain and the underlying cause thereof, e.g., inflammation. In certain embodiments, the compositions of the invention include from about 0.1 to about 20.0 percent by weight of pharmaceutically active ingredient, based on total weight of the composition. The pharmaceutically active ingredient can include diclofenac, lidocaine and mixtures thereof. Also included in the invention is the use of other acceptable forms of diclofenac and/or acceptable base forms relating to lidocaine. It has been found that the administration of diclofenac and/or lidocaine results in an anesthetic effect in a patient, and a decrease in inflammation at a target site. In certain embodiments, diclofenac, e.g., diclofenac sodium and lidocaine are combined, e.g., mixed or blended, in therapeutically effective amounts to form a composition and the composition is administered, e.g., topically, to the patient.

In certain embodiments, the pharmaceutically active ingredient may be at least partially dissolved, and in some instances completely dissolved, in an organic and/or inorganic solvent. In accordance with the invention, it is contemplated that the pharmaceutically active ingredient(s) is/are substantially uniformly distributed throughout the composition.

In certain embodiments, the compositions include from about 0.1 to about 10.0 percent by weight of a NSAID, e.g., diclofenac or diclofenac sodium, and from about 0.1 to about 10.0 percent by eight of an anesthetic, e.g., lidocaine, based on total weight of the composition.

In addition to the one or more pharmaceutically active ingredients, the compositions of the invention may also include other ingredients and additives.

The compositions of the invention further include a solvent, which can be selected from known solvents, e.g., inorganic and/or organic solvents, such as but not limited to water and/or alcohol, e.g., ethanol. In certain embodiments, the solvent is present in the compositions of the invention in an amount that constitutes from about 10.0 to about 95.0 percent by weight, based on total weight of the composition. In other embodiments, the compositions include from about 30.0 to about 60.0 percent by weight of water and from about 10.0 to about 50.0 percent by weight of alcohol, e.g., ethanol, based on total weight of the composition.

In accordance with the invention, the compositions can also include a polymer or co-polymer which can be selected from those known in the art for pharmaceutical formulations, e.g., gels. A non-limiting example of a suitable polymer is polyacrylate, e.g., carbomer, which is commercially available under the trade name carbomer 940. The polymer, e.g., Carbomer 940, can be present in an amount that constitutes from about 0.1 to about 5.0 percent by weight, based on total weight of the composition.

Other additives for use in the compositions of the invention can include diol and amine, such as propylene glycol and triethanolamine, respectively. The amount of these additives can vary and can constitute from about 1.0 to about 50.0 percent by weight, based on total weight of the composition. In certain embodiments, diol, e.g., propylene glycol, is present in an amount from about 2.0 to about 40.0 percent by weight and amine, e.g., triethanolamine, is present in an amount from about 1.0 to about 10.1, based on total weight of the composition.

Furthermore, menthol and/or methyl salicylate may be added to the compositions of the invention. When present, menthol and/or methyl salicylate typically constitute from about 0.1 to about 10.0 percent by weight, based on total weight of the composition. In certain embodiments, menthol and methyl salicylate can each constitute from about 0.1 to about 5.0 percent by weight, based on total weight of the composition.

In certain embodiments, the compositions of the invention can be prepared by dissolving the active ingredient(s) in dry, e.g. granular or powder form, in liquid solvent to form a solution and subsequently adding the remaining ingredients to the solution. The various ingredients may be added in amounts such that the resulting pharmaceutical composition is in a topical, e.g., gel, form. For example, diclofenac and lidocaine granules or powders can be added individually or as a mixture/blend to water or alcohol., e.g., ethanol, or to a mixture/blend of water and alcohol for dissolution therein. Menthol and methyl salicylate in dry form can also be added to, and dissolved in, the solvent. Thus, in certain embodiments, the solution includes diclofenac, lidocaine, menthol, methyl salicylate, water and ethanol. To the solution is then added one or more of propylene glycol, carbomer and triethanolamine to form a topical, e.g., gel, formulation.

In the embodiments wherein a gel formulation is formed, it is desired to control the amount of each of the ingredients/components such that the texture and feel (e.g., slip and drag) of the gel is appropriate for application to human skin. It is further desired to preclude a gel form that is too oily. In these embodiments, the water is present in an amount of about 10% to about 50% by weight, the alcohol, e.g., ethanol, is present in an amount of about 5% to about 50% by weight, the diol, e.g., propylene glycol, is present in an amount of about 5% to about 40% by weight and the methyl salicylate is present in an amount of about 0.01% to about 1% by weight.

In certain embodiments, the compositions may include one or more optional ingredients/additives that are selected from a wide variety that are known in the art for use in topical formulations. Nonlimiting examples include, but are not limited to, penetration enhancers, polymers, skin cooling substances and pH modifying agents. For example, in gel form, the compositions may include penetration enhancers and a hydrophilic polymer, such as but not limited to acrylic acid. The polymer can contribute to adjusting and controlling the viscosity of the gel compositions. The penetration enhancer can contribute to the ability of the gel compositions to be absorbed into the various levels or layers of the skin of the patient in order for the pharmaceutically active ingredients to reach a target site, e.g., source of inflammation.

The ingredients of the compositions can be combined, e.g., mixed or blended, at atmospheric, temperatures and conditions. Further, it is contemplated that the dry ingredients can be combined together and then, either immediately or at a subsequent time thereafter, the dry ingredients, individually or a combination thereof, can be combined with the liquid solvent and at least partially dissolved within the liquid solvent.

In accordance with the invention, the pharmaceutical compositions can be prepared/produced, distributed, delivered and administered to patients using various mechanisms. In certain embodiments, the ingredients/components which make-up the compositions are pre-combined, e.g., pre-mixed or pre-blended. For example, the ingredients/components (such as those described above) may be combined (e.g., dry ingredients added to and dissolved in water/alcohol solvent) at a manufacturing facility and then packaged for distribution and delivery. The packaged product may be provided, e.g., delivered, directly to patients in a ready-to-administer form such that the patients may self-administer the product without the need for any additional preparation. Alternatively, the product may be provided to physician offices and/or pharmacies and/or hospitals in a ready-to-administer form such that the physician offices and/or pharmacies and/or hospitals can administer the product to the patient without the need for any additional preparation. Further, the product may delivered in containers that do not include labels. Thus, the physician, pharmacist or hospital professional may affix to the containers labels that, in addition to identifying the contents of the containers, may show the name of the physician/physician's office, pharmacist/pharmacy or hospital. In contrast, it is typical for ready-to-administer pharmaceutical compositions to be labeled with the name of the manufacturer or distributor.

It is also contemplated, in accordance with the invention, that the ingredients/components which make-up the compositions may not be pre-combined, e.g., pre-mixed or pre-blended at a manufacturing facility. In these embodiments, the ingredients/components can be individually provided to the patients and/or physicians' offices and/or pharmacies and/or hospitals and the individual ingredients/components can be subsequently combined together by a patient, physician, medical professional, pharmacist and the like to form a composition in accordance with the invention. The mixing or blending of the individual ingredients/components can be conducted and the resulting composition stored for future administration or, alternately, the mixing or blending of the individual ingredients/components can be conducted at the time, or shortly before, the composition is to be administered to a patient.

In one embodiment, the individual ingredients/components that makeup the compositions are prepared and/or obtained by a supplier. The supplier then distributes and/or delivers the individual ingredients/components to a recipient, such as, a patient, a physician's office, hospital or pharmacy. The supplier further provides any apparatus that is needed to prepare the composition by combining the ingredients/components. Some ingredients may be provided in dry form, e.g., granules or powder, and other ingredients may be provided in liquid form. As previously mentioned, the dry ingredients (either individually or in the form of a mixture) can be added to the liquid ingredients, e.g., solvent, for dissolution therein. The apparatus provided by the supplier may be rented to the recipient or leased or sold. Further, it is contemplated that the use of the apparatus and the cost associated therewith may be included in the cost and purchase price of the pharmaceutical compositions. The prepared compositions are then packaged into containers and labels are affixed thereto to identify the contents of the containers, as well as, the provider of the compositions. As mentioned herein, it is customary in the art for pharmaceutical labels to include the name of the manufacturing company or facility. However, in accordance with the invention, the labels can include the name of the physician, hospital or pharmacy. In those embodiments wherein the supplier distributes and delivers the composition ingredients/components directly to the patient for preparation and self-use, a label merely identifying the contents and the name of the patient can suffice.

Without intending to be bound by any particular theory, it is believed that affixing a label to a pharmaceutical composition allows physicians, hospitals and pharmacies to develop brand recognition and loyalty with the patients that purchase and use the compositions.

Further, it is contemplated that the ingredients/components of the compositions can be provided in the form of a kit. For example, pre-packaged and pre-measured amounts or doses of each of the necessary ingredients/components may be provided individually in separate containers or vials along with instructions as to how to combine the ingredients to produce the composition. The kit can also include any utensils or equipment that is necessary to mix or blend the ingredients to form the compositions. For compositions that require complex equipment, e.g., equipment that is not compatible with packaging in a kit configuration, the equipment may be rented or leased apart from the kit. A patient, physician, pharmacist or medical professional can subsequently mix on-site the ingredients together in according to the instructions to obtain the composition. Alternately, all of the dry, e.g., powder, ingredients may be combined into one pre-packaged container or vial and the liquid ingredient or component can be contained in a separate pre-packaged container. The patient, physician, pharmacist or medical professional can subsequently mix the dry ingredients with the liquid ingredient to produce the composition as needed. These drug delivery and administration embodiments, preclude the need for the patient to visit a retail pharmacy or other related retail distribution facility.

In one embodiment, the composition of the invention is prepared in a hospital under the direct supervision of a pharmacist or physician as follows:

1. The pharmaceutically active ingredient including NSAID, anesthetic, pharmaceutically acceptable salts thereof, or mixtures thereof, and topical delivery compounds are delivered to the hospital by a supplier, e.g., FirstLine Meds;

2. An employee/representative of the supplier provides supplier-owned equipment into the facility for combining the ingredients and compounds of the composition;

3. The employee/representative of the supplier prepares the composition and fills containers with the composition; and

4. Labels identifying the hospital name are affixed to the containers for sale and distribution.

Thus, instead of the composition/container having a label which identifies the supplier or another distributor, the composition/container has a label affixed thereto which identifies the hospital that is delivering and/or administering the composition/container to the patient.

As previously identified hereto, it is contemplated that steps 2 and 3 above can be modified to include the use of a kit such that an employee/representative of the hospital can combine the components and ingredients of the composition and fill the containers therewith. Furthermore, it is contemplated that in step 2, the hospital can purchase, rent or lease from the supplier the particular equipment that is needed to combine the components and ingredients of the composition.

It is understood that the processes of providing and delivering the composition through a non-traditional sales and distribution flow path is unique and allows a facility, e.g., hospital, to label and sell the compound as its product at a price that it pre-determines or pre-selects. As a result, the facility has the ability to generate increased profits and good will based on its direct distribution to its patients.

The present invention provides new compositions for use in the treatment of pain and inflammation, as well as new methods of preparing and delivering the compositions for administration to patients, and furthermore, new methods of allowing the distributor, e.g., hospital or physician, to financially benefit by controlling the labeling and pricing of the compositions.

### EXAMPLE

A pharmaceutical composition containing one percent by weight diclofenac sodium in accordance with certain embodiments of the invention (“Invention” Formula) was prepared and compared with a product which is commercially available under the trade name Voltaren. The composition in accordance with the invention also included the following ingredients in the specified amounts.

| Ingredients | Wt % |
|---|---|
| Propylene glycol | 10.00 |
| Menthol | 0.50 |
| Methyl salicylate | 0.30 |
| Ethanol | 30.00 |
| Water | 55.45 |
| Lidocaine HCl | 0.25 |
| Carbomer 940 | 1.00 |
| Triethanolamine | 1.50 |

The diclofenac sodium, lidocaine, menthol and methyl salicylate were dissolved in a mixture of the water and ethanol to form a solution. To the solution were then added the propylene glycol, carbomer and triethanoloamine to form a gel. Each of the compositions were evaluated by in vitro flux testing using human cadaver skin.

The primary test for determining penetration of a substance from a given composition or formulation is an in vitro permeation test (“flux test”). This test evaluates the percutaneous absorption of a drug or chemical moiety across a membrane. The device employed in this example is known in the industry as a Franz diffusion cell. The cell included a donor compartment and a receptor compartment. Positioned between the compartments was a membrane. The test article was placed in the donor chamber above the membrane. The receptor chamber was filled with receiver media, and samples were taken at pre-determined intervals. Human cadaver skin and buffered saline were used as the receiver media.

Table 1 shows the amount permeated at each time point and the total amount of API delivered for each formula over the duration of the test.

TABLE 1

| | Time Point Diclofenac Sodium | | | | |
|---|---|---|---|---|---|
| | (μg/cm²/hour) | | | | Diclofenac Sodium |
| | Formula | | | | Cumulative Delivery |
| | 1 | 2 | 3 | 4 | (μg/cm²) |
| Invention | 1.35 | 2.74 | 2.50 | 0.80 | 30.41 |
| Voltaren | 1.27 | 1.78 | 1.07 | 0.34 | 15.78 |

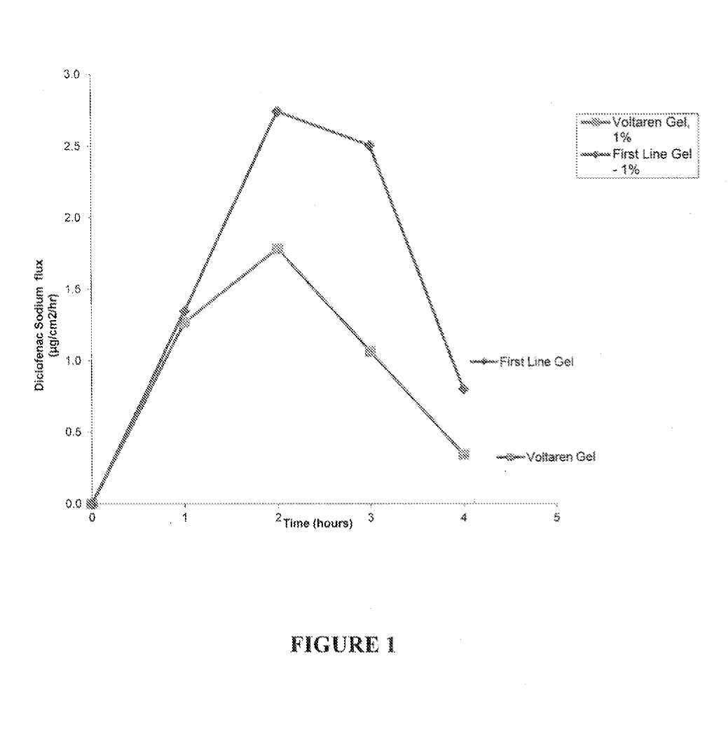

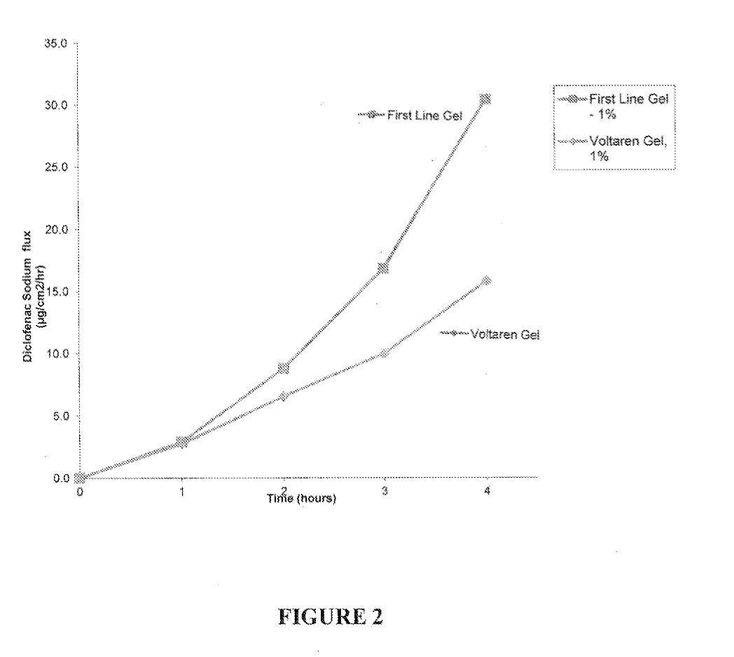

FIGS. 1 and 2 are plots showing the diclofenac sodium flux in vitro through the human epidermis over a period of time for Voltaren as compared to the gel composition of the invention. The data demonstrates that increasing the drug concentration resulted in increased delivery, e.g., from 1% to 2%.

While example systems, methods, and the like have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosed concept is not limited to the specific details, the representative apparatus, and illustrative examples shown and described. Thus, this application is intended to embrace alterations, modifications, and variations that fall within the scope of the appended claims.

We claim:

1. A pharmaceutical composition for the treatment of inflammation, comprising:
a pharmaceutically active ingredient selected from the group consisting of diclofenac and lidocaine;
solvent selected from the group consisting of water, alcohol and mixtures thereof;
diol; and
methyl salicylate.

2. The composition of claim 1, wherein the pharmaceutically active ingredient is present in an amount of about 0.1 to about 20.0 percent by weight, the water is present in an amount of about 30% to about 60% by weight, the alcohol is present in an amount of about 10% to about 50% by weight, the propylene glycol is present in an amount of about 2% to about 40% by weight and the methyl salicylate is present in the in an amount of about 01% to about 5% by weight.

3. The composition of claim 1, further comprising one or more of a penetration enhancer, polymer, skin cooling substance and pH modifying agent.

4. The composition of claim 1, wherein the pharmaceutically active ingredient is in dry form.

5. The composition of claim 1, wherein the composition is in a form for topical administration to a patient in a therapeutically effective amount.

6. The composition of claim 3, wherein the polymer is selected from the group consisting of co-polymer, acrylic acid and mixtures thereof.

7. The composition of claim 5, wherein said composition is in the form of a gel.

8. A method of providing a pharmaceutical composition for treating a patient for inflammation, comprising:
obtaining a pharmaceutically active ingredient selected from the group consisting of NSAID, anesthetic, pharmaceutically acceptable salts and forms thereof, and mixtures thereof;
obtaining a solvent;
supplying by the supplier to a recipient the pharmaceutically active ingredient and the solvent individually;
combining by the supplier or the recipient the pharmaceutically active ingredient and the solvent;
at least partially dissolving the pharmaceutically active ingredient in the solvent to form the pharmaceutical composition;
placing the pharmaceutical composition in a container;
affixing a label to the container to identify the name of the recipient; and
offering for sale and selling the container with the label to a patient.

9. The method of claim 8, wherein the pharmaceutically active ingredient is selected from the group consisting of diclofenac, lidocaine, pharmaceutically acceptable salts and forms thereof, and mixtures thereof.

10. The method of claim 8, wherein the pharmaceutical composition includes one or more ingredients selected from the group consisting of diol, amine, polymer, methyl salicylate and mixtures thereof.

11. The method of claim 8, wherein the combining includes the use of equipment that is provided by the supplier to the recipient.

12. The method of claim 11, wherein the equipment is purchased, rented or leased by the recipient from the supplier.

13. The method of claim 8, wherein the composition is in the form of a gel.

14. The method of claim 8, wherein the recipient is selected from the group consisting of a physician, physician's office, pharmacist, pharmacy and hospital.

15. A kit for providing a pharmaceutical composition for treating a patient for inflammation comprising:
one or more containers of pharmaceutically active ingredient selected from the group consisting of NSAID, anesthetic, pharmaceutically acceptable salts and forms thereof, and mixtures thereof;
one or more containers of solvent;
instructions for combining the pharmaceutically active ingredient with the solvent to form the pharmaceutical composition;
equipment for use in combining the pharmaceutically active ingredient with the solvent;
container for receiving the pharmaceutical composition;
labels for affixing to the container to identify the name of a supplier or preparer of the pharmaceutical composition.
